(19) United States
(12) Patent Application Publication
(10) Pub. No.: US 2008/0139728 A1
(43) Pub. Date: Jun. 12, 2008

(54) Composition Containing a Hydrogenated Bisglycidyl Ether and a Cross-Linking Agent

(75) Inventors: Rainer Klopsch, Bockenheim (DE); Michael Becker, Offenburg (DE); Jan-Dirk Arndt, Mannheim (DE); Frederik van Laar, Limburgerhof (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/814,005

(22) PCT Filed: Jan. 18, 2006
(86) PCT No.: PCT/EP06/00389

§ 371 Date: Jul. 16, 2007

(30) Foreign Application Priority Data
Jan. 21, 2005 (DE) 10 2005 003 116.1

(51) Int. Cl. C08G 59/24; C07D 407/02; C08G 59/40; C08L 63/02; C08K 3/34

(52) U.S. Cl. 524/442; 549/560; 525/523

(57) ABSTRACT

Composition comprising a hydrogenated bisglycidyl ether and a crosslinker, wherein the hydrogenated bisglycidyl ether has the formula I

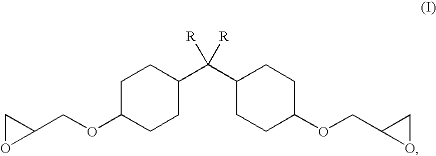

where R is CH₃ or H, and is obtained by hydrogenation of the aromatic rings of a corresponding bisglycidyl ether of the formula II

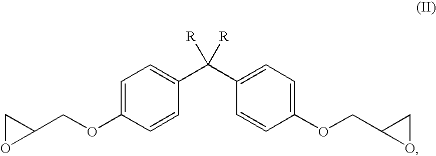

where the degree of hydrogenation is >98%, and the crosslinker has no aromatic structural elements.

Process for preparing a crosslinked epoxy resin, in which the abovementioned composition is used.

Crosslinked epoxy resins which can be prepared by the abovementioned process, and their uses.

The present invention relates to a composition comprising a hydrogenated bisglycidyl ether and a crosslinker, a crosslinked epoxy resin, a process for preparing it and its uses.

J. W. Muskopf et al. “Epoxy Resins” in Ullmann's Encyclopedia of Industrial Chemistry, 6th Edition, Vol. 12, describe, in a review, types of bisglycidyl ethers, their preparation, their reaction with various agents to form crosslinked epoxy resins and uses of these crosslinked epoxy resins.

JP-A2-11 199 645 of Jul. 27, 1999 (equivalent: U.S. Pat. No. 6,060,611) (Mitsubishi Chemical Corp.) relates to an epoxy resin composition comprising a hydrogenated epoxy resin and a crosslinker, with the epoxy resin having been prepared by hydrogenation of a corresponding aromatic epoxy resin and the degree of hydrogenation of the aromatic rings being at least 85% and the loss of epoxy groups in the hydrogenation being not more than 20%.

Crosslinkers taught are, inter alia, aromatic compounds such as phenols and imidazoles.

JP-A2-2002 037 856 of Feb. 6, 2002 (no equivalents) (Dainippon Ink, Maruzen Sekiyu) describes an epoxy resin composition comprising a hydrogenated epoxy resin and a crosslinker, with the epoxy resin having been prepared by hydrogenation of a corresponding aromatic epoxy resin and the degree of hydrogenation of the aromatic rings being at least 60%, in particular at least 90%.

Crosslinkers taught are, inter alia, aromatic novolak phenolic resins.

The German patent applications No. 10361157.6 of Dec. 22, 2003 and No. 102004055764.0 of Nov. 18, 2004 (BASF AG) relate to a heterogeneous ruthenium catalyst comprising silicon dioxide as support material, with the percentage ratio of the Q₂ and Q₃ structures Q₂/Q₃ in the silicon dioxide determined by means of solid- state ²⁹Si-NMR being less than 25, a process for preparing a bisglycidyl ether of the formula I

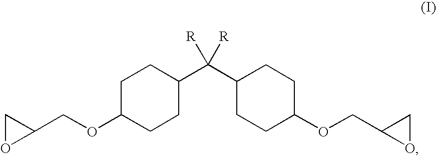

where R is CH₃ or H, by ring hydrogenation of the corresponding aromatic bisglycidyl ether of the formula II

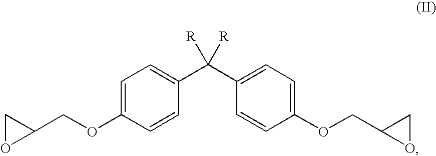

using the abovementioned heterogeneous ruthenium catalyst, and bisglycidyl ethers of the formula I which can be prepared by this process.

The German patent applications No. 10361151.7 of Dec. 22, 2003 and No. 102004055805.1 of Nov. 18, 2004 (BASF AG) relate to a heterogeneous ruthenium catalyst comprising silicon dioxide as support material, with the catalyst surface comprising alkaline earth metal ions (M²⁺), a process for hydrogenating a carbocyclic aromatic group to form the corresponding carbocyclic aliphatic group, in particular a process for preparing the bisglycidyl ethers of the formula I in which R is CH₃ or H by ring hydrogenation of the corresponding aromatic bisglycidyl ether of the formula II using the abovementioned heterogeneous ruthenium catalyst, and bisglycidyl ethers of the formula I which can be prepared by this process.

The compound II in which R=H is also referred to as bis[glycidyloxyphenyl]methane (molecular weight: 312 g/mol).

The compound II in which R=CH₃ is also referred to as 2,2-bis[p-glycidyloxiphenyl]-propane (molecular weight: 340 g/mol).

The preparation of cycloaliphatic oxirane compounds I which have no aromatic groups is of particular interest for the production of light- and weathering-resistant surface coating systems. Such compounds can basically be prepared by hydrogenation of corresponding aromatic compounds II. The compounds I are therefore also referred to as “ring-hydrogenated bisglycidyl ethers of the bisphenols A and F”.

Crosslinked epoxy resins of the prior art have a more or less high proportion of aromatic structural elements which originate from the bisglycidyl ethers used and/or the crosslinkers (hardeners) used.

It was an object of the present invention to discover improved crosslinked epoxy resins which, compared to those of the prior art, are, in particular, more light- and/or UV-stable and also have a low viscosity, minimal shrinkage and/or a very high transparency (small color number), and in their typical applications and new applications lead to improved products.

The light/UV stability is determined by the Xenotest (type 1200, BETA, Suntest) DIN EN ISO 11 341; ISO 4892-2; DIN EN ISO 11 507.

The viscosity is determined in accordance with: DIN 51 562-1; DIN 53214; DIN 53229; DIN 53018; DIN 53 019; ISO 3219.

The color number (transparency) is determined in accordance with DIN ISO 6271 (platinum-cobalt color number, APHA color number).

We have accordingly found a composition comprising a hydrogenated bisglycidyl ether and a crosslinker, wherein the hydrogenated bisglycidyl ether has the formula I

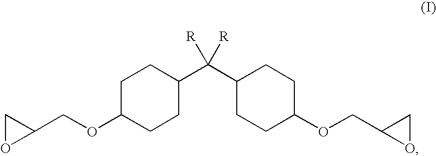

where R is CH₃ or H, and is obtained by hydrogenation of the aromatic rings of a corresponding bisglycidyl ether of the formula II

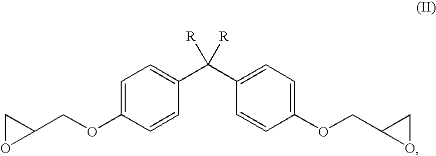

where the degree of hydrogenation is >98%, and the crosslinker has no aromatic structural elements.

Furthermore, we have found a process for preparing a crosslinked epoxy resin, in which the abovementioned composition is used.

The invention further provides crosslinked epoxy resins which can be prepared by the abovementioned process, and their uses.

A low viscosity as achieved according to the invention of the crosslinked epoxy resins found is advantageous because it makes solvent-free processing possible in its typical applications.

The light and UV stability achieved according to the invention of the crosslinked epoxy resins found makes it possible to produce yellowing-free and yellowing-stable, i.e. light and/or UV stable, materials.

The hydrogenated bisglycidyl ether I in the composition of the invention can be prepared by catalytic hydrogenation of the aromatic rings of a corresponding bisglycidyl ether of the formula II, as follows:

An important constituent of preferred hydrogenation catalysts is the support material based on amorphous silicon dioxide. In this context, the term “amorphous” means that the proportion of crystalline silicon dioxide phases in the support material is less than 10% by weight, e.g. from 0 to 8% by weight. However, the support materials used for producing the catalysts can display superstructures formed by a regular arrangement of pores in the support material.

It is preferred that the percentage ratio of the Q₂ and Q₃ structures Q₂/Q₃ determined by means of solid-state ²⁹Si-NMR is less than 25, preferably less than 20, particularly preferably less than 15, e.g. in the range from 0 to 14 or from 0.1 to 13. This also means that the degree of condensation of the silica in the support used is particularly high.

The identification of the Qₙ structures (n=2, 3, 4) and the determination of the percentage ratio is carried out by means of solid-state ²⁹Si-NMR.

Qₙ=Si(OSi)ₙ(OH)₄₋ₙ, where n=1, 2, 3 or 4.

Qₙ is found at −110.8 ppm when n=4, at −100.5 ppm when n=3 and at −90.7 ppm when n=2 (standard: tetramethylsilane) (Q₀ and Q₁ were not identified). The analysis is carried out under the conditions of “magic angle spinning” at room temperature (20° C.) (MAS 5500 Hz) with circular polarization (CP 5 ms) and using dipolar decoupling of ¹H. Owing to the partial superimposition of the signals, the intensities were evaluated by means of line shape analysis. The line shape analysis was carried out using a standard software package from Galactic Industries, with an iterative “least squares fit” being calculated.

The support material preferably comprises not more than 1% by weight, in particular not more than 0.5% by weight and particularly preferably <500 ppm by weight, of aluminum oxide, calculated as Al₂O₃.

Since the condensation of the silica can also be influenced by aluminum and iron, the total concentration of Al(III) and Fe(II and/or III) is preferably less than 300 ppm, particularly preferably less than 200 ppm, and is, for example, in the range from 0 to 180 ppm.

The Roman numerals in brackets after the element symbol indicate the oxidation state of the element.

The alkali metal oxide content generally results from the production of the support material and can be up to 2% by weight. It is frequently less than 1% by weight. Supports which are free of alkali metal oxide (from 0 to <0.1% by weight) are also suitable. The proportion of MgO, CaO, TiO₂ or ZrO₂ can amount to up to 10% by weight of the support material and is preferably not more than 5% by weight. However, support materials which comprise no detectable amounts of these metal oxides (from 0 to <0.1% by weight) are also suitable.

Since Al(III) and Fe(II and/or III) incorporated in silica can produce acid centers, it is preferred that charge-compensating cations, preferably alkaline earth metal cations (M²⁺, M=Be, Mg, Ca, Sr, Ba), are present in the support. This means that the weight ratio of M(II) to (Al(III)+Fe(II and/or III)) is greater than 0.5, preferably >1, particularly preferably greater than 3.

(M(II)=alkaline earth metal in the oxidation state 2).

Preferred support materials are amorphous silicon dioxides comprising at least 90% by weight of silicon dioxide, with the remaining 10% by weight, preferably not more than 5% by weight, of the support material also being able to be another oxidic material, e.g. MgO, CaO, TiO₂, ZrO₂, Fe₂O₃ and/or an alkali metal oxide.

In a preferred embodiment of the catalyst, the support material is halogen-free, in particular chlorine-free, i.e. the halogen content of the support material is less than 500 ppm by weight, e.g. in the range from 0 to 400 ppm by weight.

Preference is given to support materials which have a specific surface area in the range from 30 to 700 m²/g, preferably from 30 to 450 m²/g, (BET surface area in accordance with DIN 66131).

Suitable amorphous support materials based on silicon dioxide are well known to those skilled in the art and are commercially available (cf., for example, O. W. Flörke, “Silica” in Ullmann's Encyclopedia of Industrial Chemistry 6th Edition on CD-ROM). They can either be of natural origin or have been produced synthetically. Examples of suitable amorphous support materials based on silicon dioxide are silica gels and pyrogenic silica. In a preferred embodiment of the invention, the catalysts have silica gels as support materials.

Depending on the form of the preferred catalyst, the support material can have various forms. If the hydrogenation process is carried out as a suspension process, the support material will usually be used in the form of a finely divided powder for producing the catalysts. The powder preferably has particle sizes in the range from 1 to 200 μm, in particular from 1 to 100 μm. When the catalyst is used in fixed beds, it is usual to employ shaped bodies made of the support material which are obtainable, for example, by extrusion, ram extrusion or tableting and can have, for example, the shape of spheres, pellets, cylinders, extrudates, rings or hollow cylinders, stars and the like. The dimensions of these shaped bodies are usually in the range from 1 mm to 25 mm. Catalyst extrudates having extrudate diameters of from 1.5 to 5 mm and extrudate lengths of from 2 to 25 mm are frequently used.

The ruthenium content of the catalyst can be varied over a wide range. It will preferably be at least 0.1% by weight, preferably at least 0.2% by weight, and will frequently not exceed a value of 10% by weight, in each case based on the weight of the support material and calculated as elemental ruthenium. The ruthenium content is preferably in the range from 0.2 to 7% by weight, in particular in the range from 0.4 to 5% by weight, e.g. from 1.5 to 2% by weight.

The ruthenium catalysts which are preferably used in the hydrogenation process are preferably produced by firstly treating the support material with a solution of a low molecular weight ruthenium compound, hereinafter referred to as (ruthenium) precursor, in such a way that the desired amount of ruthenium is taken up by the support material. Preferred solvents here are glacial acetic acid, water or mixtures thereof. This step will hereinafter also be referred to as impregnation. The support which has been treated in this way is subsequently dried, preferably with the upper limits to the temperature indicated below being adhered to. If appropriate, the solid obtained in this way is then again treated with the aqueous solution of the ruthenium precursor and dried again. This procedure is repeated until the amount of ruthenium compound taken up by the support material corresponds to the desired ruthenium content of the catalyst.

The treatment or impregnation of the support material can be carried out in various ways and depends in a known manner on the shape of the support material. For example, the support material can be sprayed or flushed with the precursor solution or the support material can be suspended in the precursor solution. For example, the support material can be suspended in the aqueous solution of the ruthenium precursor and filtered off from the aqueous supernatant liquid after a particular time. The ruthenium content of the catalyst can be controlled in a simple fashion via the amount of liquid taken up and the ruthenium concentration of the solution. The impregnation of the support material can, for example, also be carried out by treating the support with a defined amount of the solution of the ruthenium precursor corresponding to the maximum amount of liquid which can be taken up by the support material. For this purpose, the support material can, for example, be sprayed with the required amount of liquid. Suitable apparatuses for this purpose are the apparatuses customarily used for mixing liquids with solids (cf. Vauck/Müller, Grundoperationen chemischer Verfahrenstechnik, 10th edition, Deutscher Verlag für Grundstoffindustrie, 1994, page 405 ff.), for example tumble dryers, impregnation drums, drum mixers, blade mixers and the like. Monolithic supports are usually flushed with the aqueous solutions of the ruthenium precursor.

The solutions used for impregnation are preferably low in halogen, in particular low in chlorine, i.e. they comprise no halogen or less than 500 ppm by weight of halogen, in particular less than 100 ppm by weight of halogen, e.g. from 0 to ≦80 ppm by weight of halogen, based on the total weight of the solution. Ruthenium precursors used are therefore RuCl₃ and preferably ruthenium compounds which comprise no chemically bound halogen and are sufficiently soluble in the solvent. These include, for example, ruthenium(III) nitrosyl nitrate (Ru(NO)(NO₃)₃), ruthenium(III) acetate and also alkali metal ruthenates(IV), e.g. sodium and potassium ruthenate(IV).

A very particularly preferred Ru precursor is Ru(III) acetate. This Ru compound is usually employed as a solution in acetic acid or glacial acetic acid, but it can also be used as a solid. The catalyst used according to the invention can be produced without using water.

Many ruthenium precursors are commercially available as solutions, but the corresponding solids can also be used. These precursors can be dissolved or diluted using the same component as the solvent supplied, e.g. nitric acid, acetic acid, hydrochloric acid, or preferably using water. Mixtures of water or solvent comprising up to 50% by volume of one or more organic solvents which are miscible with water or solvents, e.g. mixtures with C₁-C₄-alkanols such as methanol, ethanol, n-propanol or isopropanol, can also be used. All mixtures should be chosen so that a single solution or phase is present. The concentration of the ruthenium precursor in the solutions naturally depends on the amount of ruthenium precursor to be applied and on the uptake capacity of the support material for the solution and is preferably in the range from 0.1 to 20% by weight.

Drying can be carried out by the customary methods of solids drying with the above-mentioned upper limits to the temperature being adhered to. Adherence to the upper limit of the drying temperature is important for the quality, i.e. the activity, of the catalyst. Exceeding the drying temperatures indicated below leads to a significant loss in activity. Calcination of the support at high temperatures, e.g. above 300° C. or even 400° C., as proposed in the prior art, is not only superfluous but also has an adverse effect on the activity of the catalyst. To achieve sufficient drying rates, drying is preferably carried out at elevated temperature, preferably at ≦180° C., particularly preferably at ≦160° C., and at at least 40° C., in particular at least 70° C., especially at least 100° C., very particularly preferably at least 140° C.

Drying of the solid impregnated with the ruthenium precursor is usually carried out under atmospheric pressure, with a reduced pressure also being able to be employed to promote drying. A gas stream, e.g. air or nitrogen, will frequently be passed over or through the material to be dried in order to promote drying.

The drying time naturally depends on the desired degree of drying and on the drying temperature and is preferably in the range from 1 h to 30 h, preferably in the range from 2 to 10 h.

Drying of the treated support material is preferably carried out to the point where the content of water or of volatile solvent constituents prior to the subsequent reduction is less than 5% by weight, in particular not more than 2% by weight, based on the total weight of the solid. The proportions by weight indicated correspond to the weight loss of the solid determined at a temperature of 160° C., a pressure of 1 bar and a time of 10 minutes. In this way, the activity of the catalysts used according to the invention can be increased further.

Drying is preferably carried out with the solid which has been treated with the precursor solution being kept in motion, for example by drying the solid in a rotary tube oven or a rotary sphere oven. In this way, the activity of the catalysts used according to the invention can be increased further.

The conversion of the solid obtained after drying into its catalytically active form is achieved by reducing the solid in a manner known per se at the temperatures indicated above.

For this purpose, the support material is brought into contact with hydrogen or a mixture of hydrogen and an inert gas at the temperatures indicated above. The absolute hydrogen pressure is of minor importance for the result of the reduction and will be varied, for example, in the range from 0.2 bar to 1.5 bar. The hydrogenation of the catalyst material is frequently carried out at a hydrogen pressure of one atmosphere in a stream of hydrogen. The reduction is preferably carried out with the solid being kept in motion, for example by reducing the solid in a rotary tube oven or a rotary sphere oven. In this way, the activity of the catalysts used according to the invention can be increased further.

The reduction can also be carried out by means of organic reducing agents such as hydrazine, formaldehyde, formates or acetates.

After the reduction, the catalyst can be passivated in a known manner, e.g. by briefly treating the catalyst with an oxygen-comprising gas, e.g. air, but preferably using an inert gas mixture comprising from 1 to 10% by volume of oxygen, to improve the handlability. CO₂ or CO₂/O₂ mixtures can also be employed here.

The active catalyst can also be stored under an inert organic solvent, e.g. ethylene glycol.

Owing to the way in which the preferred catalysts are produced, the ruthenium is present in them as metallic ruthenium. In addition, electron-microscopic studies (SEM or TEM) have shown that the catalyst is a surface-impregnated catalyst: the ruthenium concentration within the catalyst particle decreases from the outside toward the interior, with a ruthenium layer being present at the surface of the particle. In the surface shell, crystalline ruthenium can be detected by means of SAD (selected area diffraction) and XRD (X-ray diffraction).

In addition, as a result of the use of halogen-free, in particular chlorine-free, ruthenium precursors and solvents in the production of the catalysts, the halide content, in particular chloride content, of the catalysts used according to the invention is below 0.05% by weight (from 0 to <500 ppm by weight, e.g. in the range 0-400 ppm by weight), based on the total weight of the catalyst.

The chloride content is, for example, determined by ion chromatography using the method described below.

In this document, all ppm figures are by weight (ppm by weight) unless indicated otherwise.

Aromatic bisglycidyl ethers of the formula II which are preferably used for the hydrogenation have a content of chloride and/or organically bound chlorine of ≦1000 ppm by weight, particularly preferably <950 ppm by weight, in particular in the range from 0 to <800 ppm by weight, e.g. from 600 to 1000 ppm by weight. The content of chloride and/or organically bound chlorine is determined, for example, by ion chromatography or coulometry using the methods described below.

According to a particular embodiment of the hydrogenation process, it has been found to be advantageous for the aromatic bisglycidyl ether of the formula II which is used to have a content of corresponding oligomeric bisglycidyl ethers of less than 10% by weight, in particular less than 5% by weight, particularly preferably less than 1.5% by weight, very particularly preferably less than 0.5% by weight, e.g. in the range from 0 to <0.4% by weight.

The oligomer content of the aromatic bisglycidyl ether of the formula II which is used is preferably determined by GPC (gel permeation chromatography) or by determination of the evaporation residue.

The evaporation residue is determined by heating the aromatic bisglycidyl ether at 200° C. for 2 hours and at 300° C. for a further 2 hours, in each case at 3 mbar.

For the further respective conditions for determining the oligomer content, see below.

The respective oligomeric bisglycidyl ethers generally have a molecular weight determined by GPC in the range from 380 to 1500 g/mol and possess, for example, the following structures (cf., for example, Journal of Chromatography 238 (1982), pages 385-398, page 387):

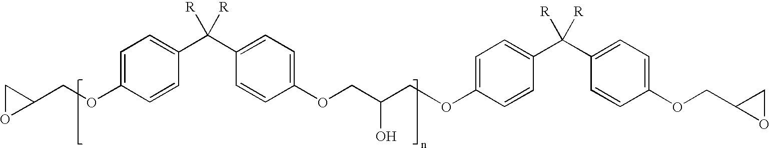

R=CH₃ or H. n=1,2,3 or 4.

The respective oligomeric bisglycidyl ethers have a molecular weight in the range from 568 to 1338 g/mol, in particular from 568 to 812 g/mol, when R=H and have a molecular weight in the range from 624 to 1478 g/mol, in particular from 624 to 908 g/mol, when R=CH₃.

The removal of the oligomers is carried out, for example, by means of chromatography or, on a relatively large scale, preferably by distillation, e.g. in a batch distillation on the laboratory scale or in a thin film evaporator, preferably in a short path distillation, on an industrial scale, in each case under reduced pressure.

In a batch distillation for the removal of oligomers at, for example, a pressure of about 2 mbar, the bath temperature is about 260° C. and the temperature at which the distillate goes over at the top is about 229° C.

The removal of the oligomers can likewise be carried out under milder conditions, for example under reduced pressures in the range from 1 to 10⁻³ mbar. At a working pressure of 0.1 mbar, the boiling point of the oligomer-comprising starting material decreases by about 20-30° C., depending on the starting material, and the thermal stress on the product thus also decreases. To minimize the thermal stress, the distillation is preferably carried out continuously in a thin film evaporation or, particularly preferably, in a short path evaporation.

In the hydrogenation process, the hydrogenation of the compounds II preferably occurs in the liquid phase. Owing to the sometimes high viscosity of the compounds II, they are preferably used as a solution or mixture in an organic solvent.

Possible organic solvents are basically those which are able to dissolve the compound II virtually completely or are completely miscible with this and are inert under the hydrogenation conditions, i.e. are not hydrogenated.

Examples of suitable solvents are cyclic and acyclic ethers, e.g. tetrahydrofuran, dioxane, methyl tert-butyl ether, dimethoxyethane, dimethoxypropane, dimethyl diethylene glycol, aliphatic alcohols such as methanol, ethanol, n-propanol or isopropanol, n-butanol, 2-butanol, isobutanol or tert-butanol, carboxylic esters such as methyl acetate, ethyl acetate, propyl acetate or butyl acetate, and also aliphatic ether alcohols such as methoxypropanol.

The concentration of compound II in the liquid phase to be hydrogenated can in principle be chosen freely and is frequently in the range from 20 to 95% by weight, based on the total weight of the solution/mixture. In the case of compounds II which are sufficiently fluid under the reaction conditions, the hydrogenation can also be carried out in the absence of a solvent.

Apart from carrying out the reaction (hydrogenation) under anhydrous conditions, it has been found to be advantageous in a number of cases to carry out the reaction (hydrogenation) in the presence of water. The proportion of water can be, based on the mixture to be hydrogenated, up to 10% by weight, e.g. from 0.1 to 10% by weight, preferably from 0.2 to 7% by weight and in particular from 0.5 to 5% by weight.

The actual hydrogenation is usually carried out by a method analogous to the known hydrogenation processes for the preparation of compounds I, as are described in the prior art mentioned at the outset. For this purpose, the compound II, preferably as a liquid phase, is brought into contact with the catalyst in the presence of hydrogen. The catalyst can either be suspended in the liquid phase (suspension process) or the liquid phase is passed over a moving bed of catalyst (moving-bed process) or a fixed bed of catalyst (fixed-bed process). The hydrogenation can be carried out either continuously or batchwise. The process of the invention is preferably carried out as a fixed-bed process in trickle-bed reactors. The hydrogen can be passed over the catalyst either in cocurrent with or in countercurrent to the solution of the starting material to be hydrogenated.

Suitable apparatuses for carrying out a hydrogenation in the suspension mode and also for hydrogenation over a moving bed of catalyst or a fixed bed of catalyst are known from the prior art, e.g. from Ullmanns Enzyklopädie der Technischen Chemie, 4th edition, volume 13, p. 135 ff., and also from P. N. Rylander, “Hydrogenation and Dehydrogenation” in Ullmann's Encyclopedia of Industrial Chemistry, 5th Ed. on CD-ROM.

The hydrogenation can be carried out either at a hydrogen pressure of one atmosphere or at a superatmospheric pressure of hydrogen, e.g. at an absolute hydrogen pressure of at least 1.1 bar, preferably at least 10 bar. In general, the absolute hydrogen pressure will not exceed a value of 325 bar and preferably 300 bar. The absolute hydrogen pressure is particularly preferably in the range from 50 to 300 bar.

The reaction temperatures are generally at least 30° C. and will frequently not exceed a value of 150° C. In particular, the hydrogenation process is carried out at temperatures in the range from 40 to 100° C. and particularly preferably in the range from 45 to 80° C.

Possible reaction gases are hydrogen and also hydrogen-comprising gases which comprise no catalyst poisons such as carbon monoxide or sulfur-comprising gases, e.g. mixtures of hydrogen with inert gases such as nitrogen or offgases from a reformer, which usually further comprise volatile hydrocarbons. Preference is given to using pure hydrogen (purity ≧99.9% by volume, particularly preferably ≧99.95% by volume, in particular ≧99.99% by volume).

Owing to the high catalyst activity, comparatively small amounts of catalyst, based on the starting material used, are required. Thus, less than 5 mol %, e.g. from 0.2 mol % to 2 mol %, of ruthenium will preferably be used per 1 mole of compound 11 in a suspension process carried out batchwise. In the case of a continuous hydrogenation process, the starting material II to be hydrogenated will usually be passed over the catalyst in an amount of from 0.05 to 3 kg/(l(catalyst)h), in particular from 0.15 to 2 kg/(l(catalyst)h).

Of course, when the activity of the catalysts used in this process drops, they can be regenerated by the customary methods known to those skilled in the art for noble metal catalysts such as ruthenium catalysts. Mention may here be made of, for example, treatment of the catalyst with oxygen as described in BE 882 279, treatment with dilute, halogen-free mineral acids as described in U.S. Pat. No. 4,072,628, or treatment with hydrogen peroxide, e.g. in the form of aqueous solutions having a concentration of from 0.1 to 35% by weight, or treatment with other oxidizing substances, preferably in the form of halogen-free solutions. The catalyst is usually rinsed with a solvent, e.g. water, after reactivation and before renewed use.

The hydrogenation process involves the hydrogenation of the aromatic rings of the starting bisglycidyl ether of the formula II

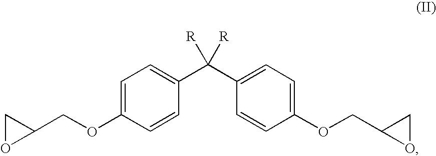

where R is CH₃ or H, with the degree of hydrogenation being >98%, particularly preferably >98.5%, very particularly preferably >99%, e.g. >99.3%, in particular >99.5%, e.g. in the range from >99.8 to 100%.

The degree of hydrogenation (Q) is defined by

Q(%)=([number of cycloaliphatic C6 rings in the product]/[number of aromatic C6 rings in the starting material])·100.

The ratio, e.g. molar ratio, of the cycloaliphatic C6 rings to aromatic C6 rings is preferably determined by means of ¹H-NMR spectroscopy (integration of the aromatic and corresponding cycloaliphatic ¹H signals).

The bisglycidyl ethers of the formula I preferably have a content of corresponding oligomeric ring-hydrogenated bisglycidyl ethers of the formula

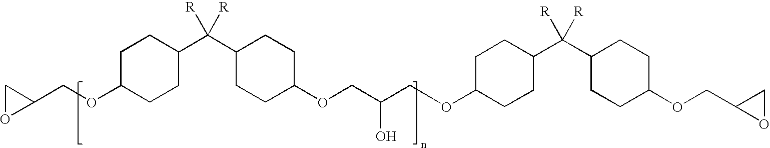

(where R is CH₃ or H) in which n=1, 2, 3 or 4, of less than 10% by weight, particularly preferably less than 5% by weight, in particular less than 1.5% by weight, very particularly preferably less than 0.5% by weight, e.g. in the range from 0 to <0.4% by weight.

The content of oligomeric ring-hydrogenated bisglycidyl ethers is preferably determined by heating the bisglycidyl ether at 200° C. for 2 hours and at 300° C. for a further 2 hours, in each case at 3 mbar, or by means of GPC (gel permeation chromatography).

For the further respective conditions for determining the oligomer content, see below.

The bisglycidyl ethers of the formula I preferably have a total chlorine content determined in accordance with DIN 51408 of less than 1000 ppm by weight, in particular less than 800 ppm by weight, very particularly preferably less than 600 ppm by weight, e.g. in the range from 0 to 400 ppm by weight.

The bisglycidyl ethers of the formula I preferably have a ruthenium content determined by mass spectrometry combined with inductively coupled plasma (ICP-MS) of less than 0.3 ppm by weight, in particular less than 0.2 ppm by weight, very particularly preferably less than 0.1 ppm by weight, e.g. in the range from 0 to 0.09 ppm by weight.

The bisglycidyl ethers of the formula I preferably have a platinum-cobalt color number (APHA color number) determined in accordance with DIN ISO 6271 of less than 30, particularly preferably less than 25, very particularly preferably less than 20, e.g. in the range from 0 to 18.

The bisglycidyl ethers of the formula I preferably have an epoxy equivalent weight determined in accordance with the standard ASTM-D-1652-88 in the range from 170 to 240 g/equivalent, particularly preferably in the range from 175 to 225 g/equivalent, very particularly preferably in the range from 180 to 220 g/equivalent.

The bisglycidyl ethers of the formula I preferably have a content of hydrolyzable chlorine determined in accordance with DIN 53188 of less than 500 ppm by weight, particularly preferably less than 400 ppm by weight, very particularly preferably less than 350 ppm by weight, e.g. in the range from 0 to 300 ppm by weight.

The bisglycidyl ethers of the formula I preferably have a kinematic viscosity determined in accordance with DIN 51562 of less than 800 mm²/s, particularly preferably less than 700 mm²/s, very particularly preferably less than 650 mm²/s, e.g. in the range from 400 to 630 mm²/s, in each case at 25° C.

The bisglycidyl ethers of the formula I preferably have a cis/cis:cis/trans:trans/trans isomer ratio in the range 44-63%:34-53%:3-22%.

The cis/cis:cis/trans:trans/trans isomer ratio is particularly preferably in the range 46-60%:36-50%:4-18%.

The cis/cis:cis/trans:trans/trans isomer ratio is very particularly preferably in the range 48-57%:38-47%:5-14%.

In particular, the cis/cis:cis/trans:trans/trans isomer ratio is in the range 51-56% :39-44%:5-10%.

The bisglycidyl ethers of the formula I are obtained by hydrogenation of the aromatic rings of a bisglycidyl ether of the formula II

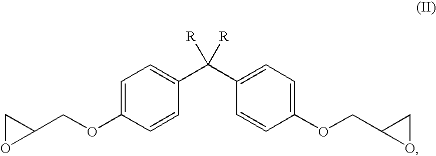

where R is CH₃ or H, with the degree of hydrogenation being >98%, particularly preferably >98.5%, very particularly preferably >99%, e.g. >99.3%, in particular >99.5%, e.g. in the range from >99.8 to 100%.

The crosslinker (hardener) in the composition of the invention:

The crosslinker has no aromatic structural elements such as aromatic C5 and/or C6 rings, in which, for example, one, two or three carbon atoms can also be replaced by heteroatoms such as N, S and/or O atoms.

As crosslinkers, it is possible to use amines, e.g. alicyclic, cyclic and polycyclic aliphatic monoamines, diamines and polyamines. Among primary, secondary and tertiary amines, the primary and secondary amines are preferred.

The low molecular weight monoamines and diamines preferably comprise pure carbon chains having 1-20 carbon atoms but can also comprise heteroatoms such as oxygen or nitrogen. The heteroatoms are preferably separated from one another by bridges comprising 2-3 carbon atoms.

Examples of monoamines and diamines as crosslinkers are:

methylamine, ethylamine, n-propylamine, isopropylamine, n-butylamine, sec-butylamine, tert.-butylamine, isopentylamine, n-hexylamine, n-octylamine, 2-ethylhexylamine, tridecylamine, dimethylamine, diethylamine, di-n-propylamine, di-n-butylamine, di-n-hexylamine, di(2-ethylhexyl)amine, ditridecylamine, hydrazine, 1,2-ethylendiamine (EDA), 1,3-propylenediamine, 1,2-propylenediamine, neopentanediamine, 1,4-butylenediamine, hexamethylenediamine, octamethylenediamine, iso-phoronediamine (IPDA), 3,3′-dimethyl-4,4′diaminodicyclohexylmethane, bis(aminomethyl)tricyclodecane (TCD-diamine, isomer mixture), 4,9-dioxadodecane-1,12-diamine (DODA), 4,7,10-trioxatridecane-1,13-diamine, 3-(methyl-amino)propylamine, 3-(cyclohexylamino)propylamine, diethylenetriamine (DETA), triethylenetetramine (TETA), tetraethylenepentamine, 3-(2-aminoethyl)-aminopropylamine, dipropylenetriamine, N,N-bis(3-aminopropyl)methylamine, N,N-dimethyldipropylenediamine, N,N′-bis(3-aminopropyl)ethylenediamine, ethanolamine, 3-amino-1-propanol, isopropanolamine, 5-amino-1-pentanol, 2-(2-aminoethoxy)ethanol, aminoethylethanolamine, N-(2-hydroxyethyl)-1,3-propanediamine, N-methylethanolamine, N-ethylethanolamine, N-butylethanolamine, diethanolamine (DEA), diisopropylamine, piperazine (PIP), N-(2-aminoethyl)piperazine, piperidine, 1,2-diaminocyclohexane, 1,3-diaminocyclohexane, 1,4-diaminocyclohexane, 1,2-diamino-3-methylcyclohexane, 1,2-diamino-4-methylcyclohexane, bis(4-aminocyclohexyl)-methane, 1,4-bis(aminomethyl)cyclohexane, m-xylylenediamine (MXDA), 3,9-bis(3-aminopropyl)-2,4,8,10-tetraspiro[5.5]undecane.

Oligodiamines and polyamines can comprise a backbone of oligo- or polyethoxylates or -propoxylates and copolymers (block copolymers or random polymers) composed of ethylene oxide (EO) and 1,2-propylene oxide (PO) (polyetheramines).

In general, aliphatic diamines and also oligoamines and polyamines whose backbone has more than 4 atoms can also comprise heteroatoms, in particular oxygen (O) and nitrogen (N) (e.g. 4,9-dioxadodecane-1,12-diamine (DODA)).

Crosslinkers can also be built up by condensation of low molecular weight amines (C₁₋₂₀-amines) with specific compounds, e.g. aldehydes to form imines.

Further crosslinkers which can be used are

carboxylic anhydrides such as maleic anhydride and succinic anhydride, with saturated anhydrides such as succinic anhydride being preferred over unsaturated anhydrides such as maleic anhydride, and cycloaliphatic acid anhydrides such as tetrahydrophthalic anhydride, methyltetrahydrophthalic anhydride, hexahydrophthalic anhydride, methylhexahydrophthalic anhydride, methyl-3,6-endomethylenetetrahydrophthalic anhydride (the Diels-Alder product of cyclopentadiene and maleic anhydride, CAS No. 25134-21-8), the anhydride of dodecenyl succinate and trialkyltetrahydrophthalic anhydride.

Polyamidoamines as are obtained, for example, from the reaction of amines with acrylic esters and subsequent reaction of the ester function with diamines can likewise be used as crosslinkers in epoxy systems. Commercially available polyamidoamines as hardeners are, for example, EPH315, 325, 340 and 345 obtainable from Bakelite or Epicure 3055, 3072 and 3090 obtainable from Resolution or H12-01, H10-25 and M947,948 obtainable from Leuna Harze.

It is likewise possible to use amino-terminated polyamides as crosslinkers. For this purpose, one of the diamines mentioned in the description is, for example, condensed with a diacid or an anhydride. Examples of such diacids and anhydrides are oxalic acid, malonic acid, succinic acid, pentanedioic acid, hexanedioic acid, octanedioic acid, dodecanedioic acid and also the above-described anhydrides and their corresponding diacids.

Furthermore, adducts of an amine or a plurality of amines with the hydrogenated bisglycidyl ether I can be used as crosslinkers.

For the present purposes, an adduct is the reaction product of an amine or a plurality of amines with a molar excess of hydrogenated bisglycidyl ether I (hBGE). The amine is an amine, in particular a primary or secondary amine, as mentioned above as crosslinker.

These crosslinkers advantageously allow the viscosities of crosslinkers and/or the composition of the invention comprising a hydrogenated bisglycidyl ether and a crosslinker to be adapted.

In a particular embodiment of the invention, SR-Dur® 2633, SR-Dur® 2485 S or SR-Dur® 2230 from SRS-Meeder GmbH, D-25836 Poppenbull, (Catalog No. 9000-1142, 9000-1147 or 9000-1029), based on aliphatic polyamine systems, are advantageously used as crosslinkers.

A mixture of two or more of the abovementioned crosslinkers can also be used as crosslinker.

Optional additives in the composition of the invention:

The composition of the invention comprising a hydrogenated bisglycidyl ether and a crosslinker can comprise additives.
(1) Pulverulent reinforcing materials and fillers, e.g. metal oxides such as aluminum oxide and magnesium oxide, metal carbonates such as calcium carbonate and magnesium carbonate, silicon compounds such as pulverulent kieselguhr, a basic magnesium silicate, calcined alumina, finely pulverulent silica, quartz and crystalline silica, metal hydroxides such as aluminum hydroxide and also kaolin, mica, quartz powder, graphite, molybdenum disulfide, etc., and likewise fibrous reinforcing materials and fillers, e.g. glass fibers, ceramic fibers, carbon fibers, aluminum fibers, silicon carbide fibers, boron fibers, polyester fibers, polyamide fibers.

These are preferably mixed in in an amount of 1-900% by weight, based on the composition comprising bisglycidyl ether and crosslinker.
(2) Colorants, pigments, flame inhibitors, e.g. titanium dioxide, iron black, molybdenum red, marine blue, ultramarine blue, cadmium yellow, cadmium red, antimony trioxide, red phosphorus, brominated compounds, triphenyl phosphate

These are preferably mixed in in an amount of 0.001-20% by weight, based on the composition comprising bisglycidyl ether and crosslinker.
(3) Furthermore, many curable monomers and oligomers and also synthetic resins can be mixed in for the purpose of improving the properties of the crosslinked epoxy resin in the final coating layers, bonding layers, shaped products, etc.

For example, one or more types of diluents for epoxy resins may be mentioned, e.g. monoepoxides, phenolic resins, aldehyde resins, melamine resins, fluorinated hydrocarbon resins, vinyl chloride resins, acrylic resins, silicone resins and polyester resins. The proportion of resins mixed in is preferably less than 50% by weight, e.g. from 1 to 45% by weight, based on the composition comprising bisglycidyl ether and crosslinker.
(4) The composition can also comprise auxiliaries such as agents for making the composition thixotropic and leveling agents.
(5) The composition can additionally comprise aliphatic reaction accelerators.

Aliphatic reaction accelerators are, for example, tertiary amines which have no aromatic structural elements and preferably no unsaturated structural elements.

Examples are 1,8-diazabicyclo(5.4.0)undec-7-ene, 1,5-diazabicyclo[4.3.0]non-5-ene, triethylenediamine (TEDA).

Aliphatic reaction accelerators also include, for example, quaternary phosphonium and ammonium salts such as tetraalkylammonium halides and tetraalkylphosphonium halides, e.g. tetrabutylammonium bromide.
(6) An additive which increases the scratch resistance of the resulting crosslinked epoxy resin is particularly advantageously added to the composition, e.g. in an amount in the range from 5 to 50% by weight based on the crosslinked epoxy resin, in particular when the crosslinked epoxy resins of the invention are used for producing scratch-resistant bodies such as automobile windows, embedding compounds for solar cells, coatings for motorbikes/mopeds/roller blades/bicycles and spectacle lenses.

An example of such an additive is nanosilicates (nanoparticles of silicates). Suitable nanosilicates from Hanse Chemie AG are named Nanopox® and are nanosilicates having a narrow distribution at diameters of less than 50 nm.

The composition of the invention can be prepared by mixing the hydrogenated bisglycidyl ether, the crosslinker(s) and optionally further components such as one or more of the abovementioned additives, e.g. the nanoparticles for producing scratch resistance.

A melt-mixing process with heating, a melt-kneading process by means of a roller or a kneading apparatus, a wet-mixing process using an appropriate solvent or a dry-mixing process can be employed for this purpose.

To prepare the crosslinked epoxy resin of the invention (a polymer), the composition of the invention comprising the hydrogenated bisglycidyl ether glycidyl ether and the crosslinker, preferably consisting of the hydrogenated bisglycidyl ether and the crosslinker and optionally one or more of the abovementioned additives, is thoroughly mixed and, depending on the components present and the desired property profile, at a temperature in the range from 15 to 250° C., e.g. from 15 to 70° C., from 60 to 120° C. or from 100 to 200° C. This reaction is referred to as curing. The curing conditions can easily be determined by a person skilled in the art as a function of the desired materials properties of the resulting epoxy resin and/or its applications.

The composition of the invention and/or the crosslinked epoxy resins of the invention are preferably used
a) for producing transparent panes and glazing, in particular for buildings, vehicles, aircraft, vision aids (spectacles) and protective devices (e.g. safety glasses, protective shields),
b) for producing materials or articles in which a structural unit or functional unit is embedded in the crosslinked epoxy resin. (Use of the crosslinked epoxy resin as embedding compound).

Examples of such materials are carbon fibers and effect products.

Examples of such articles are antenna cables and solar cells.

The encapsulation of solar cells with polymers is advantageous for reducing the production costs and for efficient sealing against environmental influences, which is indispensable for giving the solar cells a long life;cf.:

http://www.solarserver.de/solarmagazin/anlageoktober2003.html.
c) for producing transparent bowling balls. In particular, such balls have the property of enabling three-dimensional design effects to be realized on them.
d) for producing coatings for vehicles such as motorbikes, mopeds, roller blades, bicycles.
e) for producing transparent housings or transparent housing components, in particular for producing housings or housing components for electric appliances or toys, for example for producing housings or housing components for computers (desktops, laptops), printers, monitors, entertainment appliances (televisions, stereos, CD players, MP3NVMA players), communication equipment (telephones, mobile telephones, radio telephones).

### EXAMPLES

The processing of the composition of the invention to produce automobile windows, embedding compounds (e.g. for solar cells), etc., is effected by introducing the composition (reaction mixture) into a suitable mold. The composition is allowed to cure in the mold, if appropriate at elevated temperature, and the workpiece is then removed from the mold. To introduce the reaction mixture into the mold, air bubbles advantageously have to be avoided as far as possible. It is therefore advisable not to mix the reaction mixture by means of a stirrer in a vessel, but instead to homogenize it by means of a static mixer in a closed two-component machine in a manner similar to the processing of polyurethane. This method of processing makes it possible to avoid air bubbles which are undesirable in the future workpiece.

The low shrinkage of the composition of the invention and/or the crosslinked epoxy resins during curing has been confirmed in experiments in which a liquid composition according to the invention (reaction mixture) was introduced into a cylindrical mold consisting of a material which does not adhere to the resulting crosslinked epoxy resin (in this case polyethylene). The test specimen could not be removed from the cylindrical mold after curing because of its perfect fit (no shrinkage). After the mold had been cut open, the test specimen could be removed without difficulty, which demonstrates the absence of adhesion to the mold surface.

Examples of the production of hydrogenation catalysts and their use for the hydrogenation of the aromatic rings of a bisglycidyl ether of the formula II may be found in the German patent applications No. 10361157.6 of Dec. 22, 2003 and No. 102004055764.0 of Nov. 18, 2004 (BASF AG).

The conversion and the degree of hydrogenation are determined by means of ¹H-NMR: amount of sample: 20-40 mg, solvent: CDCl₃, 700 μliter using TMS as reference signal, sample tubes: 5 mm diameter, 400 or 500 MHz, 20° C.; decrease in the signals of the aromatic protons versus increase in the signals of the aliphatic protons.

### Description of the GPC Measurement Conditions

Stationary phase: 5 styrene divinylbenzene gel columns “PSS SDV linear M” (each 300×8 mm) from PSS GmbH (temperature: 35° C.).
Mobile phase: THF (flow: 1.2 ml/min.).
Calibration: MW 500-10 000 000 g/mol using PS calibration kit from Polymer Laboratories. In the oligomer range: ethylbenzene/1,3-diphenyl-butane/1,3,5-triphenylhexane/1,3,5,7-tetraphenyloctane/1,3,5,7,9-pentaphenyldecane.
Evaluation limit: 180 g/mol.
Detection: RI (index of refraction) Waters 410, UV (at 254 nm) Spectra Series UV 100.

The molecular weights reported are, owing to different hydrodynamic volumes of the individual polymer types in solution, relative values based on polystyrene as calibration substance and are thus not absolute values.

The oligomer content in % by area determined by means of GPC can be converted into % by weight by means of an internal or external standard.

GPC analysis of an aromatic bisglycidyl ether of the formula II (R=CH₃) used in the hydrogenation process of the invention indicated, for example, the following content of corresponding oligomeric bisglycidyl ethers in addition to the monomer:

Molar masses
in the range 180-<380 g/mol:>98.5% by area,
in the range 380-<520 g/mol:<1.3% by area,
in the range 520-<860 g/mol:<0.80% by area and
in the range 860-1500 g/mol:<0.15% by area.

### Description of the Method for Determining the Evaporation Residue

About 0.5 g of each sample was weighed into a weighing bottle. The weighing bottles were subsequently placed at room temperature in a plate-heated vacuum drying oven and the drying oven was evacuated. At a pressure of 3 mbar, the temperature was increased to 200° C. and the sample was dried for 2 hours. The temperature was increased to 300° C. for a further 2 hours, and the sample was subsequently cooled to room temperature in a desiccator and weighed.

The residue (oligomer content) determined by this method in standard product (ARAL-DIT GY 240 BD from Varitico) was 6.1% by weight.

The residue (oligomer content) determined by this method in distilled standard product was 0% by weight. (Distillation conditions: 1 mbar, bath temperature: 260° C., and temperature at which the distillate went over the top: 229° C.).

Determination of the cis/cis, cis/trans, trans/trans Isomer Ratios

A product output of hydrogenated bisphenol A bisglycidyl ether (R=CH₃) was analyzed by means of gas chromatography (GC and GC-MS). 3 signals were identified as hydrogenated bisphenol A bisglycidyl ether.

The hydrogenation of the bisphenol A unit of the bisglycidyl ether can result in a plurality of isomers. Depending on the arrangement of the substituents on the cyclohexane rings, cis/cis, trans/trans or cis/trans isomers can occur.

To identify the three isomers, the products corresponding to the peaks in question were collected preparatively by means of a column arrangement. Each fraction was subsequently characterized by NMR spectroscopy (¹H, ¹³C, TOCSY, HSQC).

For the preparative GC, a GC system having a column arrangement was used. In this system, the sample was preseparated on a Sil-5 capillary (I=15 m, ID=0.53 mm, df=3 μm). The signals were cut onto a 2nd GC column with the aid of a DEANS connection. This column served to check the quality of the preparative cut. Finally, each peak was collected with the aid of a fraction collector. 28 injections of an about 10% strength by weight solution of the sample were prepared, corresponding to about 10 μg of each component.

The components isolated were then characterized by NMR spectroscopy.

The isomer ratios of a hydrogenated bisphenol F bisglycidyl ether (R=H) are determined correspondingly.

### Determination of Ruthenium in the Ring-Hydrogenated Bisglycidyl Ether of the Formula I

The sample was diluted by a factor of 100 with a suitable organic solvent (e.g. NMP). The ruthenium content of this solution was determined by mass spectrometry combined with inductively coupled plasma (ICP-MS).

Instrument: ICP-MS spectrometer, e.g. Agilent 7500s

### Measurement Conditions:

Calibration: external calibration in organic matrix
Atomizer: Meinhardt
Mass: Ru102

The calibration line was selected so that the necessary release value could be determined reliably in the diluted measurement solution.

Determination of chloride and organically bound chlorine

Chloride was determined by ion chromatography.

### Sample Preparation:

About 1 g of the sample was dissolved in toluene and extracted with 10 ml of high-purity water. The aqueous phase was measured by means of ion chromatography.

### Measurement Conditions:

Ion chromatography system: Metrohm
Precolumn: DIONEX AG 12
Separation column: DIONEX AS 12
Eluent: (2.7 mmol of Na₂CO₃+0.28 mmol of NaHCO₃)/liter of water
Flow: 1 ml/min.
Detection: conductivity after chemical suppression
Suppressor: Metrohm module 753 50 mmol of H₂SO₄; high-purity water (flow: about 0.4 ml/min.)
Calibration: from 0.01 mg/l to 0.1 mg/l

Coulometric determination of organically bound chlorine (total chlorine), in accordance with DIN 51408, Part 2, “Determination of chlorine content”

The sample was burnt in an oxygen atmosphere at a temperature of about 1020° C. The bound chlorine present in the sample is in this way converted into hydrogen chloride. The nitrous gases, sulfur oxides and water formed during combustion are removed and the combustion gas which has been purified in this way is passed into the coulometer cell. Here, the chloride formed is determined coulometrically according to Cl⁻+Ag⁺→AgCl.
Sample weight range: 1 to 50 mg
Determination limit: about 1 mg/kg (substance dependent)
Instrument: Euroglas (LHG), “ECS-1200”
Reference: F. Ehrenberger, “Quantitative organische Elementaranalyse”, ISBN 3-527-28056-1.

1. A composition comprising a hydrogenated bisglycidyl ether and a crosslinker, wherein the hydrogenated bisglycidyl ether has the formula I 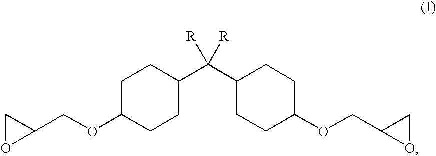
where R is CH₃ or H, and is obtained by hydrogenation of the aromatic rings of a corresponding bisglycidyl ether of the formula II 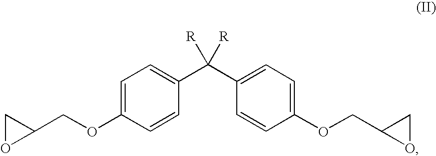
where the degree of hydrogenation is >98%, and the crosslinker has no aromatic structural elements.

2. The composition according to claim 1, wherein the degree of hydrogenation is >99%.

3. The composition according to claim 1, wherein the degree of hydrogenation is >99.5%.

4. The composition according to claim 1, wherein the hydrogenated bisglycidyl ether I has a content of corresponding oligomeric hydrogenated bisglycidyl ethers of the formula 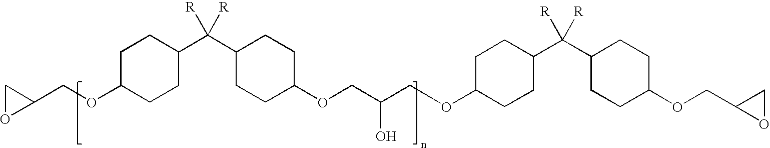
where n=1, 2, 3 or 4, of less than 10% by weight.

5. The composition according to the preceding claim claim 4, wherein the hydrogenated bisglycidyl ether I has a content of corresponding oligomeric hydrogenated bisglycidyl ethers of less than 5% by weight.

6. The composition according to claim 4, wherein the hydrogenated bisglycidyl ether I has a content of corresponding oligomeric hydrogenated bisglycidyl ethers of less than 1.5% by weight.

7. The composition according to claim 4, wherein the hydrogenated bisglycidyl ether I has a content of corresponding oligomeric hydrogenated bisglycidyl ethers of less than 0.5% by weight.

8. The composition according to claim 4, wherein the content of oligomeric hydrogenated bisglycidyl ethers is determined by heating the bisglycidyl ether I at 200° C. for 2 hours and at 300° C. for a further 2 hours, in each case at 3 mbar.

9. The composition according to claim 4, wherein the content of oligomeric hydrogenated bisglycidyl ethers is determined by means of GPC (gel permeation chromatography).

10. The composition according to claim 9, wherein the content of oligomeric bisglycidyl ethers determined in % by area by means of GPC is equated to a content in % by weight.

11. The composition according to claim 1, wherein the hydrogenated bisglycidyl ether I has a total chlorine content determined in accordance with DIN 51408 of less than 1000 ppm by weight.

12. The composition according to claim 1, wherein the hydrogenated bisglycidyl ether I has a ruthenium content determined by mass spectrometry combined with inductively coupled plasma (ICP-MS) of less than 0.3 ppm by weight.

13. The composition according to claim 1, wherein the hydrogenated bisglycidyl ether I has a platinum-cobalt color number (APHA color number) determined in accordance with DIN ISO 6271 of less than 30.

14. The composition according to claim 1, wherein the hydrogenated bisglycidyl ether I has an epoxy equivalent weight determined in accordance with the standard ASTM-D-1652-88 in the range from 170 to 240 g/equivalent.

15. The composition according to claim 1, wherein the hydrogenated bisglycidyl ether I has a content of hydrolyzable chlorine determined in accordance with DIN 53188 of less than 500 ppm by weight.

16. The composition according to claim 1, wherein the hydrogenated bisglycidyl ether I has a kinematic viscosity determined in accordance with DIN 51562 of less than 800 mm²/s at 25° C.

17. The composition according to claim 1, wherein the hydrogenated bisglycidyl ether I has a cis/cis:cis/trans:trans/trans isomer ratio in the range 44-63%:34-53%:3-22%.

18. The composition according to claim 1, wherein the crosslinker is an amine, carboxylic anhydride, polyamidoamine or adduct of an amine or a plurality of amines with the hydrogenated bisglycidyl ether I or a mixture of two or more such compounds.

19. The composition according to any claim 1, wherein the crosslinker is diethylenetriamine (DETA), triethylenetetramine (TETA), 4,9-dioxadodecane-1,12-diamine (DODA), isophoronediamine (IPDA), N-(2-aminoethyl)piperazine, dicyandiamide and/or bis(aminomethyl)tricyclodecane (TCD-diamine).

20. The composition according to claim 1, wherein the content of crosslinker is in the range from 0.01 to 200% by weight, based on the hydrogenated bisglycidyl ether I.

21. The composition according to claim 1, wherein the content of crosslinker is in the range from 0.1 to 150% by weight, based on the hydrogenated bisglycidyl ether I.

22. The composition according to claim 1, wherein the bisglycidyl ether of the formula I is obtained by hydrogenation of the aromatic rings of a bisglycidyl ether of the formula II in the presence of a heterogeneous ruthenium catalyst comprising silicon dioxide as support material, with the percentage ratio of the signal intensities of the Q₂ and Q₃ structures Q₂/Q₃ in the silicon dioxide determined by means of solid-state ²⁹Si-NMR being less than 25.

23. The composition according to claim 22, wherein the total concentration of Al(III) and Fe(II and/or III) in the silicon dioxide support material of the Ru catalyst is less than 300 ppm by weight.

24. The composition according to claim 23, wherein the silicon dioxide support material of the Ru catalyst comprises alkaline earth metal cations (M²+) in a weight ratio of
M(II):(Al(III)+Fe(II and/or III)) of >0.5.

25. The composition according to claim 1, wherein the aromatic bisglycidyl ether of the formula II used for the hydrogenation has a content of corresponding oligomeric bisglycidyl ethers of less than 10% by weight.

26. The composition according to claim 1, wherein the aromatic bisglycidyl ether of the formula II used for the hydrogenation has a content of corresponding oligomeric bisglycidyl ethers of less than 5% by weight.

27. The composition according to any of claim 1, wherein the aromatic bisglycidyl ether of the formula II used for the hydrogenation has a content of corresponding oligomeric bisglycidyl ethers of less than 1.5% by weight.

28. The composition according to claim 1 which comprises a nanosilicate.

29. A process for preparing a crosslinked epoxy resin, wherein a composition according to claim 1 is used.

30. The process according to claim 29, wherein the composition used is reacted at a temperature in the range from 20 to 250° C.

31. A crosslinked epoxy resin which can be prepared by a process according to claim 29.

32. The method of using a composition according to claim 1 and/or of a crosslinked epoxy resin for producing transparent panes and glazing.

33. The method of using according to the preceding claim for producing transparent panes and glazing for buildings, vehicles, aircraft, vision aids and protective devices.

34. The method of using a composition according to claim 1 and/or of a crosslinked epoxy resin as embedding compound.

35. The method of using a composition according to claim 1 and/or of a crosslinked epoxy resin for producing materials or articles in which a structural unit or functional unit is embedded in the crosslinked epoxy resin.

36. The use method of using according to claim 35, wherein the materials are carbon fibers or effect materials and the articles are antenna cables or solar cells.

37. The method of using a composition according to claim 1 and/or of a crosslinked epoxy resin for producing transparent bowling balls.

38. The method of using a composition according to claim 1 and/or of a crosslinked epoxy resin for producing coatings for vehicles.

39. The method of using a composition according to claim 1 and/or of a crosslinked epoxy resin for producing transparent housings or housing components.
